SEQUENCE LISTING (4 sequences)

SEQ ID NO: 1 - DNA, 50 bp, Homo sapiens
aggcgctcggggcgcaggtagggccacggctccccgcggaagatctggat

SEQ ID NO: 2 - DNA, 52 bp, Homo sapiens
ggtcaacttgtcccacttagatggcagacctgtccgacccatgcggtccgaa

SEQ ID NO: 3 - DNA, 50 bp, Homo sapiens
gggcggcagagcatccggagtggccagtcgtccctgtgtttgtgcagctg

SEQ ID NO: 4 - DNA, 52 bp, Homo sapiens
tgatgttaccagcctgagggaaggaagggttcacagcctgatatgttggtga

(19) United States
(12) Patent Application Publication
(10) Pub. No.: US 2022/0328130 A1
(43) Pub. Date: Oct. 13, 2022

(54) Opioid Receptor Score (OReS) from Combinatory Opioid Receptor Gene Polymorphisms

(71) Applicant: Jonathan Kost, Farmington, CT (US)

(72) Inventor: Jonathan Kost, Farmington, CT (US)

(21) Appl. No.: 17/138,865

(22) Filed: Dec. 30, 2020

Related U.S. Application Data
(60) Provisional application No. 62/955,305, filed on Dec. 30, 2019.

(51) Int. Cl. G16B 20/20; C12Q 1/6883; G16H 50/30; G16H 20/10

(52) U.S. Cl. CPC G16B 20/20; C12Q 1/6883; G16H 50/30; G16H 20/10; C12Q 2600/156; C12Q 2600/106

(57) ABSTRACT

Disclosed herein are compositions and methods relevant to novel Opioid Receptor Scores to determine opioid responsiveness of a human individual. The Opioid Receptor Score allows determination of innate opioid responsiveness relevant to drug treatment and can be predicted and diagnosed from blood, buccal swab or saliva. In the disclosed method, an individual is genotyped for a plurality of polymorphisms in Opioid Receptor genes μ, δ, and κ (OPRM1, OPRD1, OPRK1), encoding for Opioid Receptors mu 1, delta 1, and kappa 1. The genotypes are used to compose the Opioid Receptor Scores, which relate to the opioid responsiveness of the individual.

### SEQUENCE LISTING

The instant application contains a Sequence Listing which has been submitted in ASCII format via EFS-Web and is hereby incorporated by reference in its entirety. Said ASCII copy, created on Mar. 7, 2022, is named 01399.002US1 Sequence Listing_ST25.txt and is 866 bytes in size.

### BACKGROUND

It has become increasingly evident that genetic variants of the Opioid Receptors contribute to the development of opioid dependence. Polymorphisms in Opioid Receptor genes μ, δ and κ (OPRM1, OPRD1, OPRK1 ) have been reported to be associated with substance (drug or alcohol) dependence. The interaction of the three opioid receptors can modulate the action of opioid and non-opioid drugs. Thus, polymorphisms in OPRM1, OPRD1 and OPRK1 can jointly influence the vulnerability of individuals to drug dependence. Evidence from addiction studies also supports previous biological findings that the interaction of the three opioid receptors can modulate the action of opioid as well as non-opioid drugs and alcohol.

### BRIEF DESCRIPTION OF THE DRAWINGS

FIG. 1 illustrates Opioid Receptor Score (OReS) graphs showing a distribution and percentile ranking, respectively of OReS from a Hartford Hospital Pain Treatment Center study.

FIG. 2a illustrates the cumulative percentage vs. OReS score for a sample size of N=55 for patient ODA-21.

FIG. 2b is a chart that further details the information found in FIG. 2a as relating to interpretation of individual genotypes for the four polymorphisms of the OReS composition.

FIG. 3a illustrates the cumulative percentage vs. OReS score for a sample size of N=55 for patient ODA-22.

FIG. 3b is a chart that further details the information found in FIG. 3a. as relating to interpretation of individual genotypes for the four polymorphisms of the OReS composition.

FIG. 4 is a diagram showing high level communication interactions according to one aspect of an example of the foregoing.

FIG. 5 is a block diagram illustrating a communication system.

FIG. 6 is a flowchart showing the steps involved in implementing the communication system of FIG. 5.

### DETAILED DESCRIPTION

A common protein variant, Asn40Asp, in the μ-opioid receptor coded by an A/G polymorphism (rs1799971) in OPRM1, the respective receptor gene, has been well validated and studied. In various clinical scenarios, patients with the G risk allele (Aspartate, Asp), rather than the normal A allele (Asparagine, Asn), appeared less sensitive to opioid medications. Having at least one copy of the G allele (AG or GG) is associated with lower pain threshold and higher opioid consumption in postoperative patients.

In addition, the intronic OPRM1 polymorphism rs9479757 results in G/A transition associated with severity of heroin addiction. The rs9479757 polymorphism results in a splicing variant that skips Exon 2 and reduces transcription levels of OPRM1. In this case, the least common allele A at 8% frequency is protective. Similarly, a significant association of the δ-opioid receptor gene (OPRD1) intronic polymorphism rs2236861 with opioid dependence in European cohorts strongly indicates also an important role of this gene in dependence susceptibility. The OPRK1 polymorphism rsl 051660 and other variants in the κ-opioid receptor gene can modulate the effects of OPRM1 or OPRD1 variants, and also play a role in substance dependence with the comorbidity of depression and mood disorders.

Opioid Receptor Score (OReS)

The interactive effect of Opioid Receptors on substance dependence can be modeled with combinatorial genotyping of the genes. Genetic joint effects of Opioid Receptors are based on physiologically related susceptibilities to complex substance use disorders and dependences.

An Opioid Receptor Score (OReS) may be calculated by adding the number of risk alleles from the opioid receptors in a combinatorial system including the following four single nucleotide polymorphisms: OPRD1 rs2236861, OPRK1 rs1051660, OPRM1 rs9479757, and OPRM1 rs1799971 (see Table 1). In Table 1, the OReS ranges from 0 to 8, with an estimated mean of 3.7 based on the allelic frequencies for these 4 bi-allelic polymorphisms in the combinatorial system.

TABLE 1

| | | | Change | | Risk | |
|---|---|---|---|---|---|---|
| | | | [Minor | Structure, | allele | |
| GENE | Name | Polymorphism | Freq.] | location | (% Freq.) | Addiction |
| OPRD1 | Opioid | rs2236861 | 227 + 634 | Non- | G | Heroin, |
| | Receptor, | | G > A [0.29 | coding, | (71%) | opioids, |
| | delta 1 | | A] | Intron 1 | | alcohol |
| OPRK1 | Opioid | rs1051660 | 36 C > A | Pro12 Pro, | A | Heroin, |
| | Receptor, | | [0.08 A] | Exon 1 | (08%) | opioids, |
| | kappa 1 | | | | | alcohol |
| OPRAM1 | Opioid | rs9479757 | 84709 | Exon 2 Skip, | G | Heroin, |
| | Receptor, | | G > A [0.08 | Intron 2 | (92%) | opioids, |
| | mu 1 | | A] | | | nicotine |
| | | rs1799971 | 34162 | Asn40Asp, | G | Heroin, |
| | | | A > G | Exon 1 | (14%) | opioids |
| | | | [0.14 G] | | | |

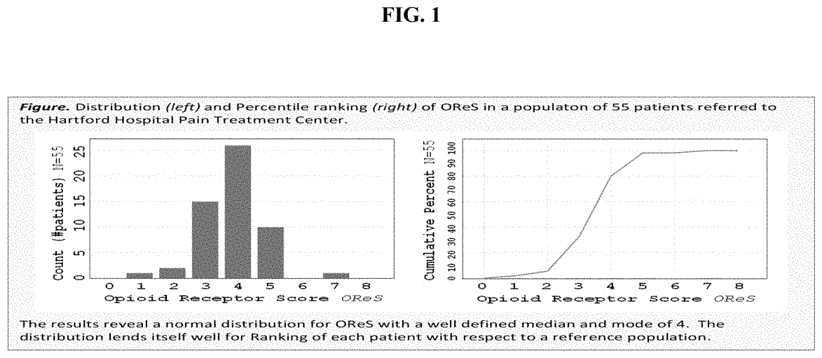

FIG. 1 illustrates Opioid receptor score graphs showing a distribution and percentile ranking, respectively of OReS from a Hartford Hospital Pain Treatment Center study. The study was conducted of feasibility of the OReS status. In a survey of 55 patients at a pain treatment center, the mean OReS was 3.84 with a median and mode of 4. OReS ranged on the low side from 0 to 3 while OReS ranged on the high side from 5 to 8 with an average value of 4 (see FIG. 1, left panel).

An OReS percentile ranking curve may be calculated from the cumulative distribution of OReS values. The median value correlates to the 50% value of the ranking curve (see FIG. 1, right panel). The ranking curve may be used to assess individual patients in a population range by placing individual OReS values in the plot.

The percentile rankings for each of the scores were estimated at higher than 80% for risk advisory and at less than 30% for a protective reassurance. Clinical correlations are drawn to those at the lower and higher ends of the distribution.

It has become increasingly evident that genetic variants of the Opioid Receptors contribute to the development of opioid dependence.

Polymorphisms in Opioid Receptor genes μ, δ and κ (OPRM1, OPRD1, OPRK1) have been reported to be associated with substance (drug or alcohol) dependence. The interaction of the three opioid receptors can modulate the action of opioid and non-opioid drugs. Thus, polymorphisms in OPRM1, OPRD1 and OPRK1 can jointly influence the vulnerability of individuals to drug dependence. Evidence from addiction studies also supports previous biological findings that the interaction of the three opioid receptors can modulate the action of opioid as well as non-opioid drugs and alcohol.

The interactive effect of Opioid Receptors on substance dependence can be modeled with combinatorial genotyping of the genes. Genetic joint effects of Opioid Receptors are based on physiologically related susceptibilities to complex substance use disorders and dependences.

The OReS components have a predetermined probability of normalizing individuals to the mean, and reducing deviation from it. In the OReS system of 4 polymorphisms, there are counterbalancing frequencies of protection and risk. This is so because the risk allele is most frequent for 2 polymorphisms in the combinatory system (OPRD1 rs2236861 G allele at 71%) and OPRM1 rs9479757 G allele at 92%) and the protective allele is most frequent for the other 2 polymorphisms (OPRK1 rs1051660 A allele at 8% and OPRM1 rs1799971 G allele at 14%). The OPRM1 gene itself, the most significant contributor to dependence, is counterbalanced by its polymorphisms rs9479757 and rs1799971, with risk alleles at 92% and 14%, respectively.

Individual genetic polymorphisms of the opioid receptors have been reported in the scientific literature (see Table 1). A common protein variant, Asn40Asp, in the μ-opioid receptor coded by an A/G polymorphism (rs1799971) in OPRM1, the respective receptor gene, has been well validated and studied. In various clinical scenarios, patients with the G risk allele (Aspartate, Asp), rather than the normal A allele (Asparagine, Asn), appeared less sensitive to opioid medications. Having at least one copy of the G allele (AG or GG) is associated with lower pain threshold and higher opioid consumption in postoperative patients. In addition, the intronic OPRM1 polymorphism rs9479757 results in G/A transition associated with severity of heroin addiction. The rs9479757 polymorphism results in a splicing variant that skips Exon 2 and reduces transcription levels of OPRM1. In this case, the least common allele A at 8% frequency is protective. Similarly, a significant association of the δ-opioid receptor gene (OPRD1) intronic polymorphism rs2236861 with opioid dependence in European cohorts strongly indicates also an important role of this gene in dependence susceptibility. The OPRK1 polymorphism rs1051660 and other variants in the κ-opioid receptor gene can modulate the effects of OPRM1 or OPRD1 variants, and also play a role in substance dependence with the comorbidity of depression and mood disorders.

### OReS ILLUSTRATIONS: CALCULATION AND CLINICAL UTILITY

### Patient ODA-21

Patient ODA-21 was a 35-year-old Hispanic female being treated for transverse myelitis, a severe auto-immune condition. She had been treated with 15 medications: acetaminophen, baclofen, buprenorphine, butalbital, cannabis (tetrahydrocannabinol), diazepam, diclofenac, duloxetine, hydrocodone, lidocaine, naloxone, oxybutynin, oxycodone, pregabalin, trazodone. The OReS genotypes for patient ODA-21 are shown in Table 2.

Patient ODA-21—OReS genotypes were as follows:

The OReS genotypes for patient ODA-21 are shown in Table 2.

TABLE 2

| | | {Risk allele} | Patient | #Risk |
|---|---|---|---|---|
| GENE | Polymorphism | Freq. | Genotype | Alleles |
| OPRD1 | rs2236861 | {G} 81% | GG | 2 |
| OPRK1 | rs1051660 | {A} 8% | AA | 2 |
| OPRM1 | rs9479757 | {G} 92% | GG | 2 |
| OPRM1 | rs1799971 | {G} 14% | AG | 1 |
| | | | OReS Value | 7 |

The regimen included 4 opioids (buprenorphine, naloxone, oxycodone, hydrocodone), to which she had not reacted well. She was then tried on Cannabis for pain relief.

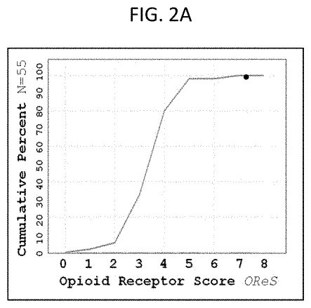

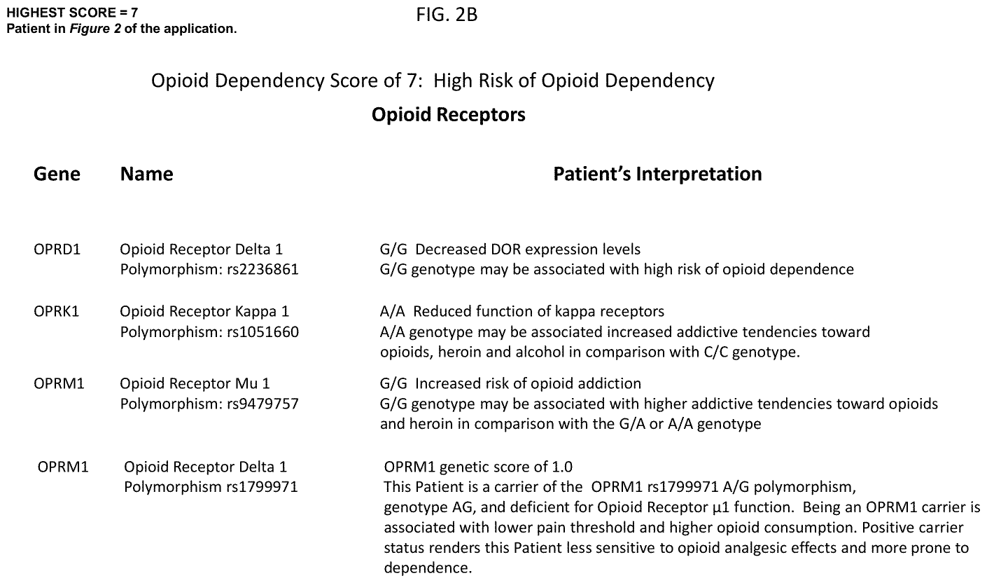

FIG. 2a illustrates the cumulative percentage vs. OReS score for a sample size of N=55 for patient ODA-21. Patient ODA-21 is at the upper 2% of the OReS ranking and opioid dependence risk, shown by the dot placement on the ranking curve. Opioids should not have been attempted, and her response confirmed the susceptibility. Cannabis, a non-opioid, is definitely a superior alternative to opioids for this patient.

Opioid receptors are commonly expressed on various immune cells, macrophages especially. These cells are prone to stimulation with opioids. This patient is at highest risk of over stimulation of the immune system to auto-immunity, which could have contributed to her transverse myelitis.

In this particular case, the patient developed a localized auto immune reaction to treatment. As such, the OReS formulations as disclosed herein may be useful in predicting adverse auto immune reactions.

FIG. 2b is a chart that further details the information found in FIG. 2a, as relating to interpretation of individual genotypes for the four polymorphisms of the opioid receptor genes. The composition OReS serves to underscore combinatory arrangements where all individual genotypes are consistently aligned to guide a patient's treatment.

### Patient ODA-22

Patient ODA-22 was a 23-year-old Caucasian female being treated for chronic back pain. She had been treated with 8 medications: buprenorphine, gabapentin, naloxone, ondansetron, oxycodone, rizatriptan, sertraline, trazodone. The regimen included 3 opioids (buprenorphine, naloxone, oxycodone) to which she had reacted well. The OReS genotypes for patient ODA-22 are shown in Table 3.

Patient ODA-22—OReS genotypes were as follows:

TABLE 3

| | | {Risk allele} | Patient | #Risk |
|---|---|---|---|---|
| GENE | Polymorphism | Freq. | Genotype | Alleles |
| OPRD1 | rs2236861 | {G} 81% | AA | 0 |
| OPRK1 | rs1051660 | {A} 8% | CC | 0 |
| OPRM1 | rs9479757 | {G} 92% | GA | 1 |
| OPRM1 | rs1799971 | {G} 14% | AA | 0 |
| | | | OReS Value | 1 |

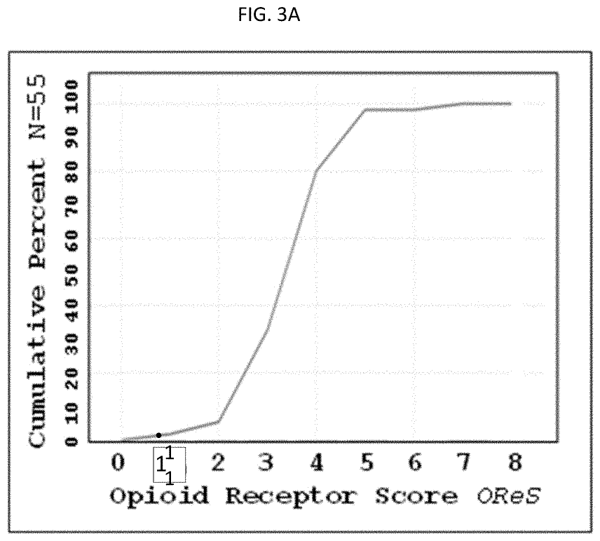

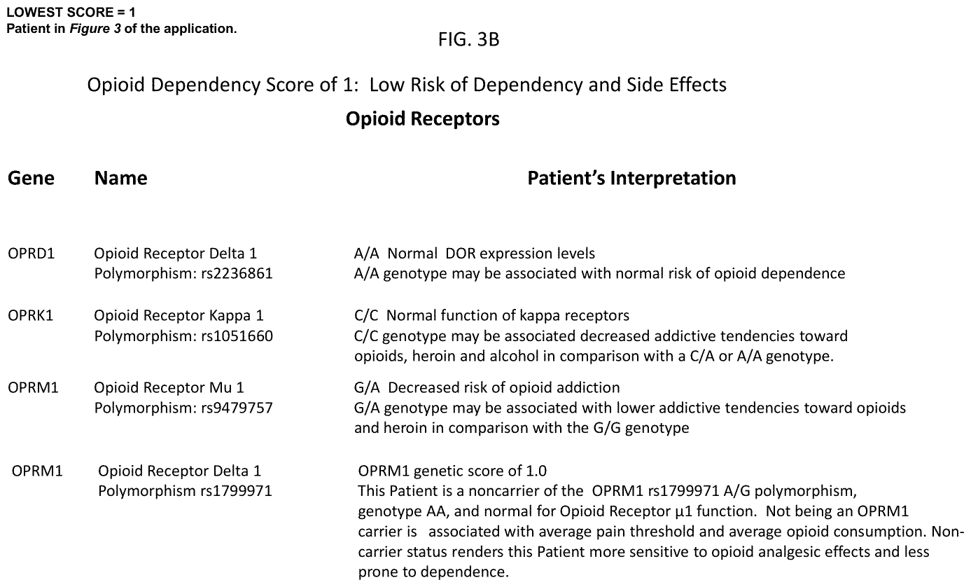

FIG. 3a illustrates the cumulative percentage vs. OReS score for a sample size of N=55 for patient ODA-22. Patient ODA-22 is at the lower 2% of the OReS ranking and opioid dependence risk. Opioids could be safely attempted, and her positive response confirmed her tolerance.

FIG. 3b is a chart that further details the information found in FIG. 3a, as relating to interpretation of individual genotypes for the four polymorphisms of the opioid receptor genes. The composition OReS serves to underscore combinatory arrangements where all individual genotypes are consistently aligned to guide a patient's treatment.

The foregoing OReS values represent linear formulations derived from adding risk alleles together. However, other formulations are contemplated including those based on non-linear formulations.

### IMPLEMENTATION

An Opioid Receptor Score (OReS) may be calculated on a mobile device or, remotely, on a server, etc. Further, the OReS may be stored locally on a mobile device or it may be stored remotely. For instance, it may be stored on a network, e.g., cloud storage, etc. Transmission of patient information, including patient medications, and patient genetic information must be securely stored and transmitted. In some examples, the patient may control access to the OReS information and genetic information using a mobile device to provide a gateway to grant permissions, access the OReS score, and access genetic information. A system implemented with the foregoing information may provide automatic updates in connection with updates to a database providing underlying information supporting the calculation of the OReS score.

Disclosed herein are compositions and methods relevant to a novel Opioid Receptor Score to determine the opioid responsiveness of a human individual. The Opioid Receptor Score allows the determination of the innate opioid responsiveness of the patient relevant to opioid treatment and can be predicted and diagnosed simply from a blood, buccal or saliva sample. In one disclosed method, an individual is genotyped for a plurality of polymorphisms in a gene encoding Opioid Receptor delta 1, a gene encoding Opioid Receptor kappa 1, and a gene encoding Opioid Receptor mu 1, and the genotypes are used to produce the Opioid Receptor Scores, which relate to the opioid responsiveness of the human individual.

Pursuit to calculating an Opiod Receptor Score, a composition of olignocleotides may be used to amplify or detect a plurality of polymorphisms in a gene OPRD1 encoding the Opioid Receptor delta 1; a gene OPRK1 encoding the Opioid Receptor kappa 1; or a gene OPRM1 encoding the Opioid Receptor mu 1. More specifically, the olignocleotides amplify or detect the following single nucleotide polymorphisms: rs1051660 (SEQ ID NO:1), rs1799971 (SEQ ID NO: 2), rs2236861 (SEQ ID NO:3), and rs9479757 (SEQ ID NO: 4). In Table 4, a sequence listing is provided for single nucleotide polymorphisms

TABLE 4
Sequence Listings for
Single Nucleotide Polymorphisms

| | | Human Variable Sequence |
|---|---|---|
| Poly- | SEQ ID | [Polymorphism] and Flanking |
| morphism | NO | Non-variable Sequences |
| rs1051660 | SEQ ID | AGGCGCTCGGGGCGCAGGTAGGGCC[ [symbol] ] |
| | NO: 1 | GGCTCCCCGCGGAAGATCTGGAT |
| rs1799971 | SEQ ID | GGTCAACTTGTCCCACTTAGATGGC[ [symbol] ] |
| | NO: 2 | ACCTGTCCGACCCATGCGGTCCGAA |
| rs2236861 | SEQ ID | GGGCGGCAGAGCATCCGGAGTGGCC[ [symbol] ] |
| | NO: 3 | TCGTCCCTGTGTTTGTGCAGCTG |
| rs9479757 | SEQ ID | TGATGTTACCAGCCTGAGGGAAGGA[ [symbol] ] |
| | NO: 4 | GGTTCACAGCCTGATATGTTGGTGA |

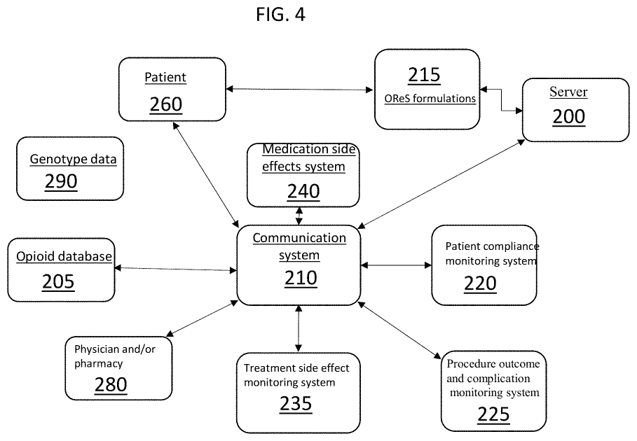

FIG. 4 is a diagram showing high level communication interactions according to one aspect of an example of the foregoing. FIG. 4 shows elements providing information to the server 200. Opioid database 205 facilitates tracking of medication and side effects, and such information is provided to the server 200. Communication system 210, which may be Health Insurance Portability and Accountability Act (HIPAA) compliant, provides email interfacing between doctor and patient, may be a source of information to server 200. A database of verified data for research 215 communicates with server 200. A patient compliance monitoring system 220 may also be in communication with server 200. A procedure outcome and complication monitoring system 225 compiles data on patient complications with medications and communicates relevant complications to server 200. A treatment side effect monitoring system 235 as well as a medication side effects system 240 provides data to server 200.

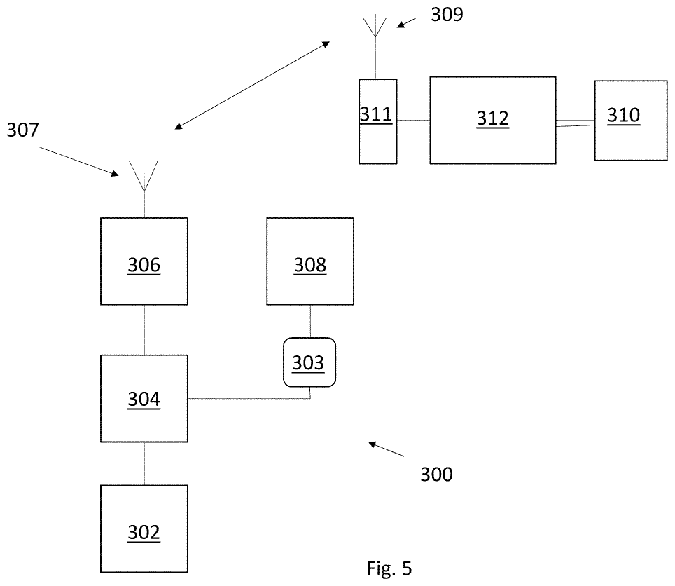

FIG. 5 is a block diagram illustrating a communication system on which the foregoing may be implemented. A user who has gone through the process of genotyping may store that genotyping data, under a protective password, within memory 302 on mobile device 300. Alternatively, the genotyping data may be stored remotely and accessed by mobile device 300. Information relating to the genotyping data (including information pertinent to determining an OReS score) may be received through transceiver 306) using antenna 307 to establish communication with a remote storage location such as server 312, through antenna 309 and communication interface 311. Transceiver 306 is also contemplated as being a separate transmitter and a separate receiver. Database 310 may contain the OReS methodology, specifically the formulations for determining the OReS value. Alternatively, or in addition thereto, database 310 may contain a pertinent OReS value and or OReS methodology. Guidance (advising as to a graded recommendation of medical usage based on the OReS value) may be provided through server 312 through communications media such as the Internet or by a corresponding communication system to transceiver 306 (which may also be a separate transmitter and receiver). The results of the guidance may be passed through transceiver 306 to processor 304 to a user interface 303 and/or shown on display 308. Alternatively, that guidance may be provided (using the Internet and or wireless link) to a treating physician, pharmacy, etc. A user interface as referenced herein may operate according to audio information (voice command, etc.).

In another example, the personal genotyping data, (e.g., OReS) may be stored in database 310 and accessed from mobile device 300, which may act as a thin client, using a password to server 312 which processes the stored personal genotyping data and genotyping information to produce guidance as determined by server 312. The guidance may be relayed using the Internet or a communication system as described above, and assessed through user interface 303 and/or shown on display 308.

In some examples, the mobile device 300 may be a wearable such as a locket or wristband. OReS information pertaining to a patient may be resident on the mobile device. Alternatively, it may be accessed remotely in connection with an RFID chip/connection, Bluetooth, Bluetooth LE, WiFi, Zigbee, radio frequency, or other forms of wireless communication. The mobile device may also hold the OReS formulation information which may be processed in connection with genotype information held in mobile device 300 or resident at a remote location, such as with a remotely-located database 310. The mobile device may be scanned, for instance, in connection with filling a prescription at a pharmacy or in connection with obtaining a prescription from a doctor.

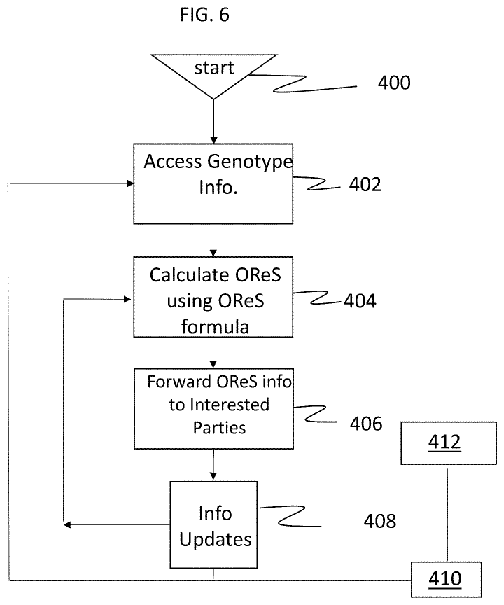

FIG. 6 is a flowchart showing the steps involved in using the foregoing. The flow starts at step 400. Genotype information is collected, in advance, from a patient and that genotype information is accessed at step 402. An OReS value is calculated for a patient at step 404. At step 406, OReS info, which may include not only the OReS value, but also the guidance (recommendations regarding the prescription in favor or against certain drugs) may be forward to interested parties (including one or more physicians, a pharmacy and the patient). A continuing flow may follow as shown in FIG. 6 where at step 408, OReS formations may be updated and/or genotype data may be updated, both of which may affect the guidance given with a particular medication or medications.

Data collected in connection with the foregoing may be used with statistical engine 410 (representing a hardware of software solution) for ranking patients according to the OReS distribution in a population. At step 412, specific advisories may be provided for patients at either the low or high end of the distribution.

The foregoing has been described herein using specific embodiments for the purposes of illustration only. Regarding the OReS value, there may be some applications where one of the components in calculated the OReS value may have a higher coefficient than others. Consequently, different algorithms and calculation of alternative OReS scores are contemplated. It will be readily apparent to one of ordinary skill in the art, however, that the principles of the foregoing can be embodied in other ways. Therefore, the foregoing should not be regarded as being limited in scope to the specific embodiments disclosed herein, but instead as being fully commensurate in scope with the following claims.

I claim:

1. A composition consisting essentially of a plurality of DNA oligonucleotides that detect or amplify a plurality of polymorphisms in a gene OPRD1 encoding the Opioid Receptor delta 1, a gene OPRK1 encoding the Opioid Receptor kappa 1, and a gene OPRM1 encoding the Opioid Receptor mu 1, the oligonucleotides amplifying or detecting the following single nucleotide polymorphisms: rs1051660 (SEQ ID NO:1); rs1799971 (SEQ ID NO:2); rs2236861 (SEQ ID NO:3); and rs9479757 (SEQ ID NO:4).

2. The composition as recited in claim 1 wherein the plurality of polymorphisms identifies a combinatory genotype for OPRD1, OPRK1 and OPRM1, for a human individual.

3. The composition as recited in claim 1 wherein each oligonucleotide comprises a detectable label.

4. The composition as recited in claim 1 wherein each oligonucleotide is attached to a solid support.

5. A system for tracking and monitoring a patient treatment, comprising:
means for storing genotype data and an Opioid Receptor Score (OReS) formulation;
means for calculating an Opioid Receptor Score (OReS) value from the OReS formulation; and
means to convey the guidance recommendation to a user and/or interested party.

6. The system for tracking and monitoring a patent treatment as recited in claim 5 wherein the interested party is selected from the group consisting of a patient, a physician, a pharmacist, a data collection site and combinations thereof

7. The system for tracking and monitoring a patent treatment as recited in claim 6 which further comprises a statistical engine for ranking patients according to the OReS distribution in a population.

8. The system for tracking and monitoring a patent treatment as recited in claim 7 which further comprises an entity for providing specific advisories to patients at either the low or high end of the OReS distribution.

9. A non-transitory, computer-readable, programmable product, for use in conjunction with a processor, comprising code, executable by the processor, to cause the processor to do the following:
receive genotype data;
access a database containing genotype information;
calculate an Opioid Receptor Score (OReS) value according to an OReS formulation.
retrieve data indicative, based on the OReS value, indicative of the advisability of prescribing certain drugs and opioid risk; and
convey the guidance recommendation to an electronic display.

10. A mobile device for providing guidance on prescription of drugs as affected by a known genotype comprising:
a memory for receiving an Opioid Receptor Score (OReS) value;
a transmitter for transmitting the OReS value to one or more remote locations;
a receiver for receiving genotype guidance based on the received OReS value and
a display for conveying the genotype guidance to a user.

11. The mobile device of claim 10 wherein the OReS is based on the number of risk alleles.

12. The mobile device of claim 11 wherein the OreS value is based on a linear formulation accounting for the number of risk alleles.

13. The mobile device of claim 11 wherein the OreS value is based on a nonlinear formulation accounting for the number of risk alleles.

14. The mobile device of claim 11 wherein the transmitter and receiver are adapted to communicate according to communication protocols consisting of an RFID, Bluetooth™, Bluetooth LE™, WiFi, Zigbee™, radio frequency, or other forms of wireless communication.

15. A mobile device for providing guidance on prescription of drugs as affected by a known genotype comprising:
a memory for storing genotype data and an Opioid Receptor Score (OReS);
a transmitter for transmitting the genotype data to a remote location;
a receiver for receiving genotype guidance based on the stored genotype data; and
a display for conveying the genotype guidance to a user.

16. The mobile device of claim 15 wherein the OReS is based on the number of risk alleles.

17. The mobile device of claim 16 wherein the OReS is based on a linear formulation accounting for the number of risk alleles.

18. The mobile device of claim 16 wherein the OReS is based on a nonlinear formulation accounting for the number of risk alleles.

19. The mobile device of claim 15 wherein the transmitter and receiver are adapted to communicate according to communication protocols consisting of an RFID, Bluetooth™, Bluetooth LE™, WiFi, Zigbee™, radio frequency, or other forms of wireless communication.
